(19) United States
(12) Patent Application Publication
(10) Pub. No.: US 2005/0084527 A1
(43) Pub. Date: Apr. 21, 2005

(54) Pharmaceutical formulations containing combinations of epinastine, pseudoephedrine, and methylephedrine

(75) Inventors: Tetsuo Hayashi, Chiba (JP); Kazuki Matsumoto, Chiba (JP); Norimitsu Umehara, Saitama (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/626,389

(22) Filed: Jul. 24, 2003

(30) Foreign Application Priority Data
Aug. 2, 2002 (EP) 02 017 409

(51) Int. Cl. A61K 31/137; A61K 9/20

(52) U.S. Cl. 424/464; 514/649

(57) ABSTRACT

A pharmaceutical composition comprising: (a) an antihistaminically-effective amount of epinastine or a pharmaceutically acceptable salt thereof; (b) a decongestant-effective amount of pseudoephedrine or a pharmaceutically acceptable salt thereof; (c) methylephedrine or a pharmaceutically acceptable salt thereof, and (d) a pharmaceutically acceptable carrier or excipient. wherein the composition does not comprise Belladonna.

### RELATED APPLICATIONS

This application claims priority to European Patent Application No. 02 017 409.0, filed Aug. 2, 2002, which is hereby incorporated by reference in its entirety.

### FIELD OF THE INVENTION

The present invention relates to novel oral pharmaceutical compositions comprising as pharmaceutically active compounds a combination of an antihistaminic-effective amount of epinastine or a pharmaceutically acceptable salt thereof and decongestant-effective amount of pseudoephedrine or a pharmaceutically acceptable salt thereof plus methylephedrine (methylephrine) or a pharmaceutically acceptable salt thereof. The formulation further comprises suitable pharmaceutically acceptable carriers or excipients. Another aspect of the present invention relates to methods for the preparation of these compositions and methods of using them in the treatment of symptoms which stem from common cold, rhinitis, rhinorrhea (nasal discharge) and nasal congestion (blocked nose), cough, sputum, allergic diseases and/or disorders like seasonal allergic rhinitis (SAR) and seasonal allergic conjunctivitis (SAC).

### BACKGROUND OF THE INVENTION

The common cold is a disease which develops various symptoms caused by contagious virus infection of nasal cavity, paranasal cavity, pharynx, or airway. In the common cold, a variety of symptoms such as rhinorrhea (nasal discharge), nasal congestion (blocked nose), sneeze, sore throat, cough, muscle pain, and headache are experienced, and the types of virus causing such symptoms are said to be more than 200. There was no direct treatment, and the only treatment is drug administration to remove or decrease the various symptoms.

On the other hand, allergy is a general term of symptoms accompanied by immunoreaction, various substance such as food, drugs, pollen, house dust, and auto emissions are often named as causative agents (allergen). Especially in recent years, seasonal allergy whose main allergen is pollen and perennial allergy whose allergen was itch and house dust have increased. Symptoms which stem from these allergies are such as nose/pharynx itch, snooze, rhinorrhea, nasal congestion, cough, asthma, eye itch, eye congestion, and foreign body feelings of the eye, and in addition to the various symptoms of the common cold. Removing the allergen is the best way as treatment, however, it is often difficult to remove the allergen completely in daily life. In recent years, desensitization therapy, which decreases the sensitivity of living body against allergen, has been tried, but patients do not completely recover and the problem that the therapy requires a long term treatment has not been resolved. Consequently, the drug administration to remove or decrease the various symptoms is the presently prevailing treatment.

It is desirable for a drug for common cold or allergic disease treatment to remove or decrease these various symptoms, but such a remedy has ever been unknown.

For example, H1 antihistaminics are effective to relieve the symptoms such as snooze and itch, but it is not necessarily effective to remove or decrease the symptoms such as nasal congestion, rhinorrhea, eye itch, and cough.

A medical composition with inhibitory effect on overactive airway secretory gland function such as rhinorrhea comprising an anticholinergic drug and an H1 antihistaminic drug is disclosed in JPA10298107.

Another medical composition with effect on nasal congestion comprises loxoprofen and an H1 antihistaminic drug and is disclosed by JPA2001-199882.

WO 98/06394 discloses a composition of H1 antihistaminic drug and an H3 antihistaminic drug.

WO 99/32125 discloses such a composition of a leukotriene antagonist and an antihistaminic drug.

These compositions try to treat the symptoms which stem from common cold or allergic diseases, although the symptoms are not yet treated optimally. In particular, this is true for symptoms which stem from common cold, rhinitis, or allergic diseases like nasal congestion and frequently coughing.

Therefore it is an objective of the present invention to develop a pharmaceutical composition which can remove or decrease these symptoms caused by common cold or allergic diseases.

### SUMMARY OF THE INVENTION

It now was found that the combination of epinastine, an H1 antihistaminic agent with antitussive activity, and the decongestants pseudoephedrine and methylephedrine successfully treat the symptoms of the abovementioned diseases.

It is one objective of the present invention to treat the symptoms of cough and cold diseases and allergic rhinitis or conjunctivitis, i.e., sneezing, itching, blocked nose, runny nose cough, and all together.

Another objective is to develop a suitable pharmaceutical formulation for treating congested Eustachian tubes and/or the airways of the respiratory system.

Another objective of the present invention is the treatment of common cold and in the symptomatic relief associated with cough, cold, and flu symptoms.

Still another objective of the present invention is to overcome the disadvantages of the medications known in the art in the treatment of SAR and/or SAC.

### DETAILED DESCRIPTION OF THE INVENTION

The present invention solves the aforementioned problems of the state of the art formulations of insufficient treatment of the aforementioned diseases by providing a pharmaceutical formulation comprising an antitussive-effective amount of epinastine or a pharmaceutically acceptable salt thereof and of a decongestant-effective amount of pseudoephedrine or a pharmaceutically acceptable salt thereof in combination with methylephedrine or a pharmaceutically acceptable salt thereof. Further ingredients of the formulation of the present invention may be pharmaceutically acceptable carriers or excipients.

Epinastine, 3-amino-9,13b-dihydro-1H-dibenz(c,f)imidazo(5,1-α)azepine, is an H1 antihistaminic active compound. For medical purpose, it is usually used as hydrochloride salt, but the present invention is also related to other pharmacologically permissive acid-additions salts or the free base. Epinastine had not yet been shown to have strong treatment effects on rhinorrhea, nasal congestion, and cough.

Methylephedrine is one of many alkaloids contained in ephedra and has sympathetic nerve stimulant action. The term methylephedrine comprises the dl form and l form, and any of them can be used for the present invention. Besides, if pharmacologically permissive salts such as methylephedrine hydrochloride is used, the effect is not different.

Pseudoephedrine used for the present invention is also contained in ephedra and also has sympathetic nerve stimulant action. The term pseudoephedrine comprises the d form, l form, and dl form and the stereoisomer, and any of them can be used for the present invention. Besides, if pharmacologically permissive salts such as pseudoephedrine hydrochloride and pseudoephedrine sulfate are used, the effect is not different.

Surprisingly, it was found out that a composition composed of epinastine, methylephedrine and pseudoephedrine is highly effective on decreasing rhinorrhea and nasal congestion, symptoms which stem from common cold or allergic diseases.

Furthermore, it was found out that the compositions composed of epinastine, methylephedrine, and pseudoephedrine, additionally was also effective in treating cough.

According to the invention, the term pharmaceutically acceptable or permissive salts means acid addition salts of the active compounds pseudoephedrine, epinastine, and/or methylephedrine. These acid addition salts can be formed with inorganic acids like hydrochloric acid, hydrobromic acid, or sulfuric acid or with organic acids such as oxalic acid, fumaric acid, or methanesulfonic acid. Epinastine is preferably used as its hydrochloric acid addition salt. Pseudoephedrine and also methylephedrine are preferably used as the hydrochlorides or the sulfates. Within the present invention, the hydrochloride salts for the latter two compounds are most preferred.

In context of the present invention, epinastine or its pharmacologically permissive salts may be blended with the other active ingredients in an amount of 2 mg to 25 mg as daily dosage for adults, 4 mg to 20 mg is more preferable, and 5 mg to 10 mg is most preferable.

The amount of methylephedrine or its pharmacologically permissive salts is 10 mg to 240 mg as daily dosage for adults, 25 mg to 150 mg is more preferable, and 50 mg to 110 mg is most preferable.

The amount of pseudoephedrine or its pharmacologically permissive salts is 10 mg to 300 mg as daily dosage for adults, 25 mg to 250 mg is more preferable, and 100 mg to 240 mg is more preferable.

Although he abovementioned active ingredients are the preferred ones and, as a consequence thereof, the formulation preferably does not contain any further active ingredients, the formulation of the present invention is not limited to these active ingredients alone. As an additional active compound, the compositions according to the invention may optionally contain one or several compounds selected from the group consisting of antipyretic and analgesic drugs such as acetaminophen, aspirin, and ethenzamide; nonsteroidal anti-inflammatory agents such as indomethacin, diclofenac sodium, ibuprofen, ketoprofen, and piroxicam; antiallergic/antihistaminic agents other than epinastine such as diphenhydramine hydrochloride, chlorpheniramine maleate, diphenylpyraline hydrochloride, and promethazine hydrochloride; cough suppressants such as dihydrocodeine phosphate, codeine phosphate, noscapine, pentoxyverine citrate, and dextromethorphan hydrobromide; expectorant drugs such as bromhexine hydrochloride, ambroxol hydrochloride, carbocysteine, and acetylcysteine; anticholinergic drugs such as isopropamide iodide and flutropium bromide; vitamins such as retinol, thiamine hydrochloride, riboflavin sodium phosphate, pyridoxine hydrochloride, cyanocobalamin, ascorbic acid, cholecalciferol, tocopherol acetate, and nicotinamide; antacids such as magnesium carbonate, aluminum sulfate, and magnesium aluminometasilicate; and crude drugs such as radix puerariae, licorice, cassia, and bupleurum root.

In a preferred embodiment, the present invention relates also to an oral pharmaceutical composition. It is preferred that the composition of the present invention is prepared for oral administration formulation. Such formulations can be manufactured by methods well known in the state of the art and comprise tablets, granules, fine granules, powders, capsules, chewable tablets, goumis, drops, foaming agents, resolvents in mouth, dry syrup and so on. Due to the short-lasting effects of pseudoephedrine and methylephedrine and, relative to this, the long-lasting effect of epinastine, it might be of advantage to have a sustained release of pseudoephedrine and/or methylephrine and an immediate release of an antihistaminic effective amount of epinastine. The preferred dosage forms are tablets or capsules.

The composition also may comprise additives. In case of solid formulation, the additives may be selected from the group of: excipients such as lactose, starch, sugar, mannitol, and microcrystalline cellulose; binding agents such as hydroxypropylcellulose, hydroxypropylmethylcellulose, gelatine, and PVP; disintegrating agents such as carboxymethylcellulose calcium and low substituted hydroxypropylcellulose; and lubricants such as magnesium stearate, cured ricinus, and talc. Other than the above, solubilizing agents, buffers, preservatives, perfumes, pigments, corrigents, and so on are can be used if necessary. Other additives that may be used are mentioned in this description.

Concerning the application via a tablet, in the context of the present the invention, a bilayer tablet might be of advantage. In such a bilayer tablet there may be a first layer A which provides for the sustained release of methylephedrine and pseudoephedrine or a pharmaceutically acceptable salt thereof, which are comprised in a decongestant effective amount. A second layer B provides for the immediate release of epinastine and comprises an antihistaminic effective amount of epinastine or a pharmaceutically acceptable salt thereof. Both layers A or B may further comprise pharmaceutically acceptable excipients and/or carriers.

The bilayer tablet according to the invention may additionally contain a tablet coating C consisting of pharmaceutically acceptable excipients, which mask the bitter taste of one of the active compounds.

In a preferred embodiment of the invention, bilayer tablet layer A comprises a decongestant effective amount of pseudoephedrine or a pharmaceutically acceptable salt thereof and methylephedrine or a pharmaceutical acceptable salt thereof in a matrix of a swellable hydrophilic polymer which provides a sustained release profile in a period of 3 hours to 24 hours, preferably 6 hours to 18 hours, most preferably about 12 hours.

In another application form, the inventive composition may be formulated as a capsule. Such a capsule can provide the active ingredients either instantly or some of them are provided instantly and others are provided in a sustained manner. As outlined above, it is preferred to formulate the active ingredients pseudoephedrine (or its salts) and methylephrine (or its salts) as a sustained releases form and epinastine or its salts as immediate release form.

Preferably the capsules are made of materials that at least partially can be digested by humans. Such capsules, for example, are disclosed in EP 0143524 which discloses a two-part capsule of material which is easily digestible by humans.

EP 0460921 describes capsules of chitosan and starch, grain powder, oligosaccharides, methacrylic acid-methylacrylate, methacrylic acid-ethylacrylate, and hydroxypropylmethylcellulose acetate, hydroxypropylmethylcellulose succinate, or hydroxypropylmethylcellulose phthalate.

GB 938828 discloses capsules comprising water-soluble gelatine, methylcellulose, polyvinylalcohol, or water-soluble non-toxic thermoplasts.

EP 0 606 486 B1 discloses capsules composed of hydroxypropylmethylcellulose, methylcellulose, hydroxypropylcellulose, starch, hydroxypropyl starch, and sodium alginate.

Principally all these capsules can be take for the present invention, preferred are gelatine-capsules, in particular hard-gelatine capsules. Other preferred capsules are made of starch or of a cellulose-derivative like hydroxypropylmethylcellulose.

Preferred standard capsules have the following physical characteristics:

STANDARD CAPSULE PHYSICAL CHARACTERISTICS

| | Capsule Size | | | | | |
|---|---|---|---|---|---|---|
| | 5 | 4 | 3 | 2 | 1 | 0 |
| Filling weight | 65 | 100 | 150 | 185 | 250 | 340 |
| (mg) | | | | | | |

Outside diameter (mm)

| | | | | | | |
|---|---|---|---|---|---|---|
| Cap | 4.89 | 5.31 | 5.82 | 6.35 | 6.90 | 7.63 |
| Body | 4.66 | 5.06 | 5.56 | 6.07 | 6.61 | 7.32 |

Length (mm) [±0.3 mm]

| | | | | | | |
|---|---|---|---|---|---|---|
| Cap | 6.05 | 7.47 | 8.23 | 9.17 | 10.01 | 11.18 |
| Body | 9.40 | 12.34 | 13.61 | 15.24 | 16.71 | 18.72 |
| Body volume | 0.13 | 0.21 | 0.28 | 0.37 | 0.49 | 0.68 |
| (mL) | | | | | | |
| Capsule weight | 28.1 | 40.0 | 50.7 | 65.2 | 76.0 | 99.0 |
| (mg) (±10%) | | | | | | |

Among them, capsule size 1 or 2 is preferred.

In case of a sustained release formulation, it is preferred that the release of pseudoephedrine and methylephedrine takes place over 3 hours to 24 hours, preferably 6 hours to 24 hours, most preferably about 12 hours to 24 hours. The preferred dose regimen is a “once a day application” regardless of how the formulation is applied.

In case of a bilayer tablet, each layer is in contact with each other in a portion of their surface, but provides independent release profiles for both active substances mentioned before. The sustained release layer A comprises, beside the active ingredient(s), a swellable hydrophilic polymer. Typical swellable hydrophilic polymers include cellulose ethers such as methylcellulose, hydroxypropylcellulose, hydroxypropylmethylcellulose, hydroxymethylcellulose, hydroxyethylcellulose, carboxymethylcellulose, and carboxyethylcellulose or mixtures thereof. The use of hydroxypropylmethylcellulose (HPMC) is preferred. Particularly useful are the HPMC polymers HPMC USP2910 and USP2208, such as METHOCEL® E5, E4M, E15M, K15M, and K100M supplied by the Dow Chemical Company. In the aforementioned abbreviations the designation “E” refers to USP2910 whereas “K” refers to USP2208. The number designation refers to the viscosity in a 2% aqueous solution (e.g., 5 designates a viscosity of 5 cps; 15M designates a viscosity of 15000 cps).

The excipients that could be optionally used in the sustained release layer A are insoluble polymers, soluble or insoluble fillers, antiadherents, coloring agents, lubricants, and additional binders. Typical fillers are, for example, lactose, microcrystalline cellulose, dibasic calcium phosphate, and cornstarch. Examples of antiadherents, which are used to prevent tablets from sticking to the tablet press, are colloidal silicon dioxide and talc. Magnesium stearate, talc, and stearic acid are typical lubricants. Typical binders are povidone and cornstarch.

The immediate release matrix layer B comprises, beside the active ingredient(s), different combinations of excipients. The excipients that could be optionally used in the immediate release layer B are insoluble polymers, soluble or insoluble fillers, antiadherents, lubricants, coloring agents, disintegrants, and additional binders. Typical fillers are, for example, lactose, microcrystalline cellulose, dibasic calcium phosphate, and cornstarch. Examples of antiadherents, which are used to prevent tablets from sticking to the tablet press, are colloidal silicon dioxide and talc. Typical disintegrants are crospovidone, sodium starch glycolate, and croscarmellose sodium. Typical coloring agents are selected from FD&C red 40 HT aluminum lake, 2-hydroxy-1,1′-azonaphthalene-3,6,4′-trisulfonic acid trisodium salt, erythrosine, iron oxides, 1-(4-sulfo-1-naphthylazo)-2-naphthol-6,8-disulfonic acid trisodium salt, 2′,4′,5′,7′-tetrabromo-4,5,6,7-tetrachlorofluorescein disodium salt, 2,4,5,7-tetraiodo-3,6-dihydroxyxanthene-9-spiro-1′-(4′,5′,6′,7′-tetrachloro-3′H-isobenzofuran-3′-one dipotassium salt, trisodium 3-carboxy-5-hydroxy-1-p-sulfophenyl-4-p-sulfophenylazopyrazole, 6-hydroxy-5-(4-sulfonphenyl)azo-2-naphthalenesulfonic acid disodium salt, and optionally aluminum lakes thereof. Magnesium stearate, talc, and stearic acid are typical lubricants. Typical binders are povidone and cornstarch.

Water and ethanol are examples of volatile components which can be used in the manufacture process of both layers to granulate powders. These volatile components are removed during processing and therefore do not appear in the finished product.

The tablet coating is optional since the presence of it does not modify significantly the release rates of the active substances present in the core layers. The presence of the coating is preferred because it masks the bitter taste of one of the active substances and enhances the properties of dosage form. Accordingly, a lot different coatings with different polymers and plasticizers, and other excipients could be used with the condition of not modifying significantly the release profile of the active substances present in the core tablet. A typical coating comprises a polymer such as hydroxypropylmethylcellulose and a plasticizer such as polyethylene glycol. Optional excipients could be added to the coating like antifoaming agents and opacifying agents.

An example of an antifoaming agent is silicone. Examples of opacifying agents are titanium dioxide, talc, and aluminum lake dyes.

The inventive formulation also can be applied via a tablet comprising sustained release and non-sustained release granules or a capsule comprising the same.

In case of such a tablet, non-sustained release granules and sustained release granules, which are coated with a sustained release film, are mixed with suitable excipients and then they are compressed as a tablet. The preferred ratio of the non-sustained release granules and the sustained release granules is 1:9 to 9:1, preferably 3:7 to 7:3.

Similarly, non-sustained release granules and sustained release granules which are coated with sustained release film are filled into a capsule. The preferred ratio of the non-sustained release granules and the sustained release granules is 1:9 to 9:1, preferably 3:7 to 7:3.

A non-sustained release granule comprises an amount of epinastine or a pharmaceutically acceptable salt thereof. Optionally it may comprises a portion of the total amount of pseudoephedrine or a pharmaceutically acceptable salt thereof and/or of the total amount of methylephedrine or a pharmaceutically acceptable salt thereof, if necessary.

A sustained release granule comprises either a portion or the total amount of pseudoephedrine or a pharmaceutically acceptable salt thereof and methylephedrine or a pharmaceutically acceptable salt thereof.

Preferably the non-sustained release granules contain only epinastine or a pharmaceutically acceptable salt thereof as active ingredient while the sustained release granules comprise the remaining active ingredients.

Any compounds conventionally used as a sustained-release coat can be used for the purpose of this invention. Specific examples which can be given include water insoluble polymers such as ethylcellulose, aminoalkyl methacrylate copolymer polyvinyl acetate, polyvinyl chloride, polyethylene, and the like; intestinally soluble polymers such as cellulose acetate phthalate, hydroxypropyl methylcellulose phthalate, hydroxypropyl methylcellulose acetate succinate, carboxymethylethylcellulose, styrene acrylic acid copolymer, methacrylic acid copolymer, maleic anhydrous acid copolymer, shellac, and the like; paraffin waxes such as paraffin, microcrystalline wax, and the like; higher alcohols, preferably saturated and unsaturated C₆-C₂₆-alcohols, preferred unbranched and unsubstituted, such as stearyl alcohol, cetyl alcohol, and the like; esters of higher fatty acids, preferably saturated and unsaturated C₆-C₂₆-acids, preferred unbranched and unsubstituted, such as glycerin fatty acid esters, hydrogenated oils, carnauba wax, beeswax, Japan (haze) wax, and the like; and higher fatty acids as defined above such as stearic acid, palmitic acid, myristic acid, behenic acid, and the like (or the sodium, calcium, or magnesium salts of these higher fatty acids).

Furthermore, the excipients that could be optionally used in sustained release film are water soluble polymers, sugar alcohols, plasticizers, titanium oxide, talc, coloring agents and so on. Typical water soluble polymers and sugar alcohols are hydroxypropyl methylcellulose, hydroxypropylcellulose, methylcellulose, polyvinylpyrrolidone, and polyethylene glycol. Typical plasticizers are glycerin fatty acid ester, triethyl citrate, propylene glycol, and triacetin.

For any of the inventive application forms, bilayer tablet or other sustained release tablet, instant release tablet, or capsule any of the aforementioned ingredients can be taken, if appropriate.

In the context of the present invention, capsules and tablets comprising sustained release and non-sustained release granules are preferred.

Preferably, the composition of the present invention does not comprise Belladonna. Under the term Belladonna are meant Belladonna alkaloids, a term which is commonly used in pharmaceutics. The exact method of their winning and the active ingredients of this mixture of alkaloids can be taken from the Deutsches Arzneibuch 9 (DAB 9), Volume 2, pages 932 to 944, Wissenschaftliche Verlagsgesellschaft Stuttgart mbH; Govi-Verlag GmbH, Frankfurt. These pages 932 to 944 are herewith incorporated by reference. Belladonna alkaloids are won as an extract of the plant Atropa belladonna, i.e., an extract of the leaves and/or the root. The main component of the Belladonna alkaloids is atropine. Atropine itself comprises L-(−)-hyoscamine and its racemate which develops by drying. Other alkaloids found in Belladonna are L-(−)-hyoscine (L-(−)-scopolamine), N-oxides of hyoscine and/or hyoscamine, atropamine, belladonnine, and optionally nicotine, N-methylpyrroline, N-methylpyrrolidine, pyridine, cuskhygrine, and further alkaloids. The names of the alkaloids as written above are taken from the German textbook DAB 9, referred to above. In case of ambiguities, the names shall be taken directly from the textbook, page 934.

If in the context of the present invention, the term glycerol esters of fatty acids are mentioned, any esters of fatty acids and glycerol or polyglycerol and theirs derivatives are meant. They include glycerol ester of acetic acid, lactic acid, citric acid, succinic acid, and diacetyltartaric acid. They also include polyglycerol ester of recinoleic acid.

In case of any doubts of the meaning of an ingredient, the definition of the Japanese Pharmacopoeia and if not defined there, the definition of the Japanese Standards of Food Additives shall apply.

### EXAMPLES

The invention will be further described by the following examples. These examples disclose certain preferred embodiments of the invention. The methods of manufacturing the compositions according to the invention, for example, granulation, tablet compression, tablet-coating, etc. are well known to the person skilled in the art. Those skilled in the art will appreciate that various changes, modifications, and substitutions can be made therein without departing from the spirit of the invention. Accordingly, it is intended that the invention be not limited to the following explicitly disclosed examples.

EXAMPLE 1

| Ingredient | Amount (g) |
|---|---|
| epinastine hydrochloride | 15 |
| methylephedrine hydrochloride | 150 |
| pseudoephedrine hydrochloride | 275 |
| lactose | 275 |
| microcrystalline cellulose | 270 |
| magnesium stearate | 15 |

The ingredients are mixed evenly, and 220 mg of the mixed powder obtained is filled in a capsule.

EXAMPLE 2

| Ingredient | Amount (g) |
|---|---|
| epinastine hydrochloride | 18 |
| methylephedrine hydrochloride | 160 |
| pseudoephedrine hydrochloride | 275 |
| anhydrous caffeine | 125 |
| lactose | 360 |
| microcrystalline cellulose | 300 |
| magnesium stearate | 12 |

The ingredients are mixed evenly, and 250 mg of the mixed powder obtained is pressed as tablet by direct compression method.

EXAMPLE 3

| Ingredient | Amount (g) |
|---|---|
| ibuprofen | 240 |
| isopropamide iodide | 4 |
| epinastine hydrochloride | 6 |
| methylephedrine hydrochloride | 36 |
| pseudoephedrine hydrochloride | 100 |
| noscapine hydrochloride | 12 |
| anhydrous caffeine | 40 |
| lactose | 80 |
| microcrystalline cellulose | 76 |
| magnesium stearate | 6 |

The ingredients are mixed, and 300 mg of the mixed powder obtained are pressed as tablet by direct compression method.

EXAMPLE 4

| Ingredient | Amount (g) |
|---|---|
| acetaminophen | 160 |
| dihydrocodeine phosphate | 8 |
| epinastine hydrochloride | 4 |
| methylephedrine hydrochloride | 20 |
| pseudoephedrine hydrochloride | 60 |
| anhydrous caffeine | 24 |
| vitamin B1 nitrate | 8 |
| vitamin C | 100 |
| corn starch | 70 |
| lactose | 80 |
| microcrystalline cellulose | 60 |
| magnesium stearate | 6 |

The ingredients are mixed, and 300 mg of the mixed powder obtained are pressed as tablet by direct compression method.

In any of the following examples the amounts of epinastine, pseudoephedrine, and methylephedrine can be adjusted to the amounts according to Examples 1 to 4.

EXAMPLE 5
Sustained Release Bilayer Tablet

mg per tablet

Core
A. First Layer: Pseudoephedrine and Methylephedrine Layer

| | |
|---|---|
| pseudoephedrine hydrochloride | 30.00 |
| methylephedrine hydrochloride | 30.00 |
| METHOCEL ® K15M PRCR* | 99.00 |
| lactose monohydrate | 52.4 |
| microcrystalline cellulose | 53.00 |
| colloidal silicon dioxide | 0.825 |
| magnesium stearate | 1.375 |
| povidone | 8.4 |
| Total First Layer | 275 |

B. Second Layer: Epinastine Layer

| | |
|---|---|
| epinastine HCl | 5.00 |
| FD&C red 40 HT aluminum lake (allura red AC) | 0.19 |
| microcrystalline cellulose | 35.00 |
| lactose monohydrate | 77.31 |
| povidone | 6.25 |
| magnesium stearate | 1.25 |
| Total Second Layer | 125 |
| Total Core | 400 |

Coating
C. Film Coating

| | |
|---|---|
| METHOCEL ® E5 | 7.50 |
| polyethylene glycol 6000 | 0.985 |
| silicone antifoam S184 | 0.015 |
| Total Film Coating | 8.50 |
| Total Film Coated Tablets | 408.50 |

*PR means Premium grade and CR means Controlled Released grade.

Method of Manufacture

A. First Layer
A1. Dissolve povidone in a hydroalcoholic mixture;
A2. Blend pseudoephedrine hydrochloride, methylephedrine hydrochloride, a portion of the microcrystalline cellulose, lactose, and METHOCEL® K15M for 5 to 30 minutes in a suitable mixer.
A3. Use alcoholic or hydroalcoholic solution prepared previously in step A1 to granulate the powder mix of step A2.
A4. Dry and mill the granulation from step A3, using suitable size screen.
A5. Blend the screened granulation with a portion of the microcrystalline cellulose and colloidal silicon dioxide for 3 to 15 minutes.
A6. Add magnesium stearate and blend for 3 to 15 minutes.

B. Second Layer
B1. Pass through a suitable screen epinastine HCl, Allura red AC (FD&C red 40 HT) aluminum lake, and microcrystalline cellulose. Blend for 5 to 30 minutes in a suitable mixer.
B2. Add lactose and povidone. Blend for 15 to 120 minutes in a suitable mixer.
B3. Add magnesium stearate. Blend for 3 to 20 minutes in a suitable mixer.

C. Compression:

Compress A and B into a suitable bilayer tableting machine in suitable size tablets.

D. Coating
D1. Dissolve METHOCEL® E5 and polyethylene glycol in suitable amount of water.
D2. Dissolve silicone antifoam in suitable amount of isopropyl alcohol.
D3. Add D2 to D1 and mix.

D4. Coat tablets with the METHOCEL® E5/polyethylene glycol solution from step D3 in a suitable coater.

EXAMPLE 6
Sustained Release Bilayer Tablet

mg per tablet

Core
A. First Layer: Pseudoephedrine and Methylephedrine Layer

| | |
|---|---|
| pseudoephedrine hydrochloride | 30.00 |
| methylephedrine hydrochloride | 30.00 |
| METHOCEL ® K15M PRCR* | 99.00 |
| lactose monohydrate | 63.10 |
| microcrystalline cellulose | 50.15 |
| colloidal silicon dioxide | 1.375 |
| magnesium stearate | 1.375 |
| Total First Layer | 225 |

B. Second Layer: Epinastine Layer

| | |
|---|---|
| epinastine HCl | 5.00 |
| microcrystalline cellulose | 84.20 |
| lactose monohydrate | 35.00 |
| Punceau 4R red aluminum lake | 0.175 |
| magnesium stearate | 0.625 |
| Total Second Layer | 125 |
| Total Core | 400 |

Coating
C. Film Coating

| | |
|---|---|
| METHOCEL ® E5 | 2.21 |
| polyethylene glycol 6000 | 1.36 |
| talc | 4.38 |
| titanium dioxide | 0.55 |
| Total Film Coating | 8.50 |
| Total Film Coated Tablets | 408.50 |

*PR means Premium grade and CR means Controlled Released grade.

Method of Manufacture

A. First Layer
A1. Blend pseudoephedrine hydrochloride, methylephedrine hydrochloride, microcrystalline cellulose, lactose, colloidal silicon dioxide, and METHOCEL® K15M for 5 to 30 minutes in a suitable mixer.
A2. Add magnesium stearate and blend for 3 to 15 minutes.

B. Second Layer
B1. Pass through a suitable screen epinastine HCl and microcrystalline cellulose. Blend for 5 to 30 minutes in a suitable mixer.
B2. Add lactose. Blend for 15 to 120 minutes in a suitable mixer.
B3. Add magnesium stearate. Blend for 3 to 20 minutes in a suitable mixer.

C. Compression:

Compress A and B into a suitable bilayer tableting machine in suitable size tablets.

D. Coating
D1. Dissolve METHOCEL® E5 and polyethylene glycol in suitable amount of water.
D2. Dissolve titanium dioxide and talc in suitable amount of water and mix.
D3. Add D2 to D1 and mix.

D4. Coat tablets with the METHOCEL® E5/polyethylene glycol solution from step D3 in a suitable coater.

EXAMPLE 7
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K4M PRCR* | 123.75 |
| lactose monohydrate | 83.00 |
| talc | 5.5 |
| magnesium stearate | 2.75 |
| Total First Layer | 275.00 |

*PR means Premium grade and CR means Controlled Released grade.

In Example 7, the second layer and coating are identical to that of Example 6 and the manufacture method was conducted analogously to the method outlined in Example 6.

EXAMPLE 8
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M PRCR* | 99.00 |
| lactose monohydrate | 49.60 |
| microcrystalline cellulose | 49.90 |
| colloidal silicon dioxide | 1.375 |
| povidone | 13.75 |
| magnesium stearate | 1.375 |
| Total First Layer | 275.00 |

*PR means Premium grade and CR means Controlled Released grade.

In Example 8, the second layer and coating are identical to that of Example 5 and the manufacture method was conducted analogously to the method outlined in Example 5.

EXAMPLE 9
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 165.00 |
| lactose | 41.75 |
| talc | 5.50 |
| magnesium stearate | 2.75 |
| Total First Layer | 275.00 |

*CR means Controlled Released grade.

In Example 9, the second layer and coating are identical to that of Example 5 and the manufacture method was conducted analogously to the method outlined in Example 5.

EXAMPLE 10
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 137.50 |
| microcrystalline cellulose | 69.25 |
| talc | 5.50 |
| magnesium stearate | 2.75 |
| ethanol | s.q. |
| Total First Layer | 275.00 |

*CR means Controlled Released grade.

In Example 10, the second layer and coating are identical to that of Example 5 and the manufacture method was conducted analogously to the method outlined in Example 5.

EXAMPLE 11
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 107.65 |
| dibasic calcium phosphate | 54.10 |
| ethylcellulose | 20.00 |
| talc | 5.50 |
| magnesium stearate | 2.75 |
| ethanol | s.q. |
| Total First Layer | 250.00 |

*CR means Controlled Released grade.

In Example 11, the second layer and coating are identical to that of Example 5 and the manufacture method was conducted analogously to the method outlined in Example 5.

EXAMPLE 12
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 68.90 |
| METHOCEL ® K100M CR* | 68.75 |
| lactose | 69.10 |
| talc | 5.50 |
| magnesium stearate | 2.75 |
| ethanol | s.q. |
| Total First Layer | 275.00 |

*CR means Controlled Released grade.

In Example 12, the second layer and coating are identical to that of Example 5 and the manufacture method was conducted analogously to the method outlined in Example 5.

EXAMPLE 13
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K100M CR* | 137.65 |
| lactose | 69.25 |
| talc | 5.50 |
| magnesium stearate | 2.75 |
| ethanol | s.q. |
| Total First Layer | 275.00 |

*CR means Controlled Released grade.

In Example 13, the second layer and coating are identical to that of Example 5 and the manufacture method was conducted analogously to the method outlined in Example 5.

EXAMPLE 14
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 103.25 |
| METHOCEL ® K100M CR* | 34.40 |
| lactose | 69.10 |
| talc | 5.50 |
| magnesium stearate | 2.75 |
| ethanol | s.q. |
| Total First Layer | 275.00 |

*CR means Controlled Released grade.

In Example 14, the second layer and coating are identical to that of Example 5 and the manufacture method was conducted analogously to the method outlined in Example 5.

EXAMPLE 15
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 117.65 |
| dibasic calcium phosphate | 54.10 |
| ethylcellulose | 10.00 |
| talc | 5.50 |
| magnesium stearate | 2.75 |
| ethanol | s.q. |
| Total First Layer | 250.00 |

*CR means Controlled Released grade.

In Example 15, the second layer and coating are identical to that of Example 5 and the manufacture method was conducted analogously to the method outlined in Example 5.

EXAMPLE 16
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 127.65 |
| lactose | 19.85 |
| microcrystalline cellulose | 34.25 |
| talc | 5.50 |
| magnesium stearate | 2.75 |
| ethanol | s.q. |
| Total First Layer | 250.00 |

*CR means Controlled Released grade.

In Example 16, the second layer and coating are identical to that of Example 5 and the manufacture method was conducted analogously to the method outlined in Example 5.

EXAMPLE 17
Sustained Release Bilayer Tablet
Core
A. First Layer: Pseudoephedrine and Methylephedrine Layer

| | mg per tablet |
|---|---|
| pseudoephedrine hydrochloride | 30.00 |
| methylephedrine hydrochloride | 30.00 |
| METHOCEL ® K15M CR* | 127.65 |
| Dibasic calcium phosphate | 54.10 |
| Talc | 5.50 |
| Magnesium Stearate | 2.75 |
| Ethanol | s.q. |
| Total First Layer | 250.00 |

*CR means Controlled Released grade.

In Example 17, the second layer and coating are identical to that of Example 5 and the manufacture method was conducted analogously to the method outlined in Example 5.

EXAMPLE 18
mg per 2 capsules

(a) Non-Sustained Release Granules: 2 Caspusles (Size 1)

| | |
|---|---|
| epinastine hydrochloride | 10.00 |
| pseudoephedrine hydrochloride | 18.00 |
| methylephedrine hydrochloride | 18.00 |
| hydroxypropylcellulose | 3.59 |
| sucrose | 499.41 |
| Non-Sustained Release Granules Total | 549.00 |

(b) Sustained Release Granules: 2 Capsules (Size 1)

| | |
|---|---|
| pseudoephedrine hydrochloride | 42.00 |
| methylephedrine hydrochloride | 42.00 |
| hydroxypropylcellulose | 4.00 |
| sucrose | 67.00 |
| methacrylic acid copolymer, type B | 40.60 |
| glycerol esters of fatty acids | 3.10 |
| talc | 1.30 |
| Sustained Release Granules Total | 200.00 |

Encapsulation Mixture

| | |
|---|---|
| non-sustained release granules | 549.00 |
| sustained release granules | 200.00 |
| talc | 1.00 |
| Total Capsules | 750.00 |

Method of Manufacture

A. Non-sustained Release Granules
A1. Dissolve hydroxypropylcellulose in ethanol.
A2. Blend epinastine hydrochloride, pseudoephedrine hydrochloride, and methylephedrine hydrochloride in a suitable mixer and pulverize the powder mix.
A3. Produce spherical granules by spraying the solution prepared previously in step A1 over sucrose, introducing the powder mix obtained from step A2.
A4. Dry and pass through granules from step A3 with suitable size screen to produce non-sustained release granules.

B. Sustained Release Granules
B1. Dissolve hydroxypropylcellulose in ethanol.
B2. Blend pseudoephedrine hydrochloride and methylephedrine hydrochloride in a suitable mixer.
B3. Produce spherical granules by spraying the solution prepared previously in step B1 over sucrose, introducing the powder mix obtained from step B2.
B4. Dry and pass through granules from step B3 with suitable size screen
B5. Dissolve methacrylic acid copolymer, type B in ethanol and mix with glycerol esters of fatty acids and talc.
B6. Coat the granules obtained from step B4 with the solution prepared previously in step B5 to produce sustained release granules.

C. Encapsulation Mixture
C1. Mix non-sustained release granules and sustained release granules with talc.

C2. Fill the mixture obtained from step C1 into capsules.

EXAMPLE 19
mg per 2 capsules

(a) Non-Sustained Release Granules: 2 Caspusles (Size 1)

| | |
|---|---|
| epinastine hydrochloride | 10.00 |
| pseudoephedrine hydrochloride | 18.00 |
| methylephedrine hydrochloride | 18.00 |
| hydroxypropylcellulose | 3.59 |
| sucrose | 499.41 |
| Non-Sustained Release Granules Total | 549.00 |

(b) Sustained Release Granules: 2 Capsules (Size 1)

| | |
|---|---|
| pseudoephedrine hydrochloride | 42.00 |
| methylephedrine hydrochloride | 42.00 |
| hydroxypropylcellulose | 4.00 |
| sucrose | 67.00 |
| ethylcellulose | 38.75 |
| hydroxypropylmethylcellulose 2910 | 1.00 |
| glycerol esters of fatty acids | 2.25 |
| talc | 3.00 |
| Sustained Release Granules Total | 200.00 |

Encapsulation Mixture

| | |
|---|---|
| non-sustained release granules | 549.00 |
| sustained release granules | 200.00 |
| talc | 1.00 |
| Total Capsules | 750.00 |

In Example 19, the manufacture method was conducted analogously to the method outlined in Example 18.

EXAMPLE 20
mg

(a) Non-Sustained Release Granules

| | |
|---|---|
| epinastine hydrochloride | 10.00 |
| pseudoephedrine hydrochloride | 18.00 |
| methylephedrine hydrochloride | 18.00 |
| hydroxypropylcellulose | 12.59 |
| microcrystalline cellulose | 178.91 |
| lactose | 12.50 |
| Non-Sustained Release Granules Total | 250.00 |

(b) Sustained Release Granules

| | |
|---|---|
| pseudoephedrine hydrochloride | 42.00 |
| methylephedrine hydrochloride | 42.00 |
| hydroxypropylcellulose | 4.00 |
| sucrose | 67.00 |
| methacrylic acid copolymer, type B | 30.45 |
| magnesium stearate | 10.15 |
| glycerol esters of fatty acids | 3.10 |
| talc | 1.30 |
| Sustained Release Granules Total | 200.00 |

Compression Mixture

| | |
|---|---|
| non-sustained release granules | 250.00 |
| sustained release granules | 200.00 |
| microcrystalline cellulose | 126.00 |
| croscarmellose sodium | 12.00 |
| talc | 1.00 |
| magnesium stearate | 6.00 mg |
| Total | 600.00 |

Method of Manufacture

A. Non-Sustained Release Granules
A1. Dissolve hydroxypropylcellulose in ethanol.
A2. Blend epinastine hydrochloride, pseudoephedrine hydrochloride, methylephedrine hydrochloride, microcrystalline cellulose, and lactose in a suitable mixer and knead the mixture with the solution from step A1.
A3. Dry and pass through granules obtained from step A2 with suitable size screen to produce non-sustained release granules.

B. Sustained Release Granules
B1. Dissolve hydroxypropylcellulose in ethanol.
B2. Blend pseudoephedrine hydrochloride and methylephedrine hydrochloride in a suitable mixer.
B3. Produce spherical granules by spraying the solution prepared previously in step B 1 over sucrose, introducing the powder mix obtained from step B2.
B4. Dry and pass through granules from step B3 with suitable size screen.
B5. Dissolve methacrylic acid copolymer, type B in ethanol and mix with glycerol esters of fatty acids, magnesium stearate, and talc.
B6. Coat the granules obtained from step B4 with the solution prepared previously in step B5 to produce sustained release granules.

C. Compression
C1. Mix non-sustained release granules and sustained release granules with microcrystalline cellulose, croscarmellose sodium, talc, and magnesium stearate.

C2. Compress the mixture into a suitable tableting machine in suitable size tablets.

EXAMPLE 21
mg per 2 capsules

(a) Non-Sustained Release Granules: 2 Caspusles (Size 1)

| | |
|---|---|
| epinastine hydrochloride | 10.00 |
| pseudoephedrine hydrochloride | 24.00 |
| methylephedrine hydrochloride | 24.00 |
| hydroxypropylcellulose | 3.59 |
| sucrose | 487.41 |
| Non-Sustained Release Granules Total | 549.00 |

(b) Sustained Release Granules: 2 Capsules (Size 1)

| | |
|---|---|
| pseudoephedrine hydrochloride | 36.00 |
| methylephedrine hydrochloride | 36.00 |
| hydroxypropylcellulose | 4.00 |
| sucrose | 79.00 |
| methacrylic acid copolymer, type B | 40.60 |
| glycerol esters of fatty acids | 3.10 |
| talc | 1.30 |
| Sustained Release Granules Total | 200.00 |

Encapsulation Mixture

| | |
|---|---|
| non-sustained release granules | 549.00 |
| sustained release granules | 200.00 |
| talc | 1.00 |
| Total Capsules | 750.00 |

In Example 21, the manufacture method was conducted analogously to the method outlined in Example 18.

EXAMPLE 22
mg per 2 capsules

(a) Non-Sustained Release Granules: 2 Caspusles (Size 1)

| | |
|---|---|
| epinastine hydrochloride | 10.00 |
| pseudoephedrine hydrochloride | 24.00 |
| methylephedrine hydrochloride | 24.00 |
| hydroxypropylcellulose | 3.59 |
| sucrose | 487.41 |
| Non-Sustained Release Granules Total | 549.00 |

(b) Sustained Release Granules: 2 Capsules (Size 1)

| | |
|---|---|
| pseudoephedrine hydrochloride | 36.00 |
| methylephedrine hydrochloride | 36.00 |
| Hydroxypropylcellulose | 4.00 |
| Sucrose | 79.00 |
| ammonio methacrylate copolymer | 40.60 |
| Glycerol esters of fatty acids | 3.10 |
| Talc | 1.30 |
| Sustained Release Granules Total | 200.00 |

Encapsulation Mixture

| | |
|---|---|
| non-sustained release granules | 549.00 |
| sustained release granules | 200.00 |
| talc | 1.00 |
| Total Capsules | 750.00 |

In Example 22, the manufacture method was conducted analogously to the method outlined in Example 18.

EXAMPLE 23
mg per 2 capsules

(a) Non-Sustained Release Granules: 2 Caspusles (Size 1)

| | |
|---|---|
| epinastine hydrochloride | 10.00 |
| pseudoephedrine hydrochloride | 24.00 |
| methylephedrine hydrochloride | 24.00 |
| hydroxypropylcellulose | 3.59 |
| sucrose | 487.41 |
| Non-Sustained Release Granules Total | 549.00 |

(b) Sustained Release Granules: 2 Capsules (Size 1)

| | |
|---|---|
| pseudoephedrine hydrochloride | 36.00 |
| methylephedrine hydrochloride | 36.00 |
| hydroxypropylcellulose | 4.00 |
| sucrose | 79.00 |
| ethylcellulose | 38.75 |
| hydroxypropylmethylcellulose 2910 | 1.00 |
| glycerol esters of fatty acids | 2.25 |
| talc | 3.00 |
| Sustained Release Granules Total | 200.00 |

Encapsulation Mixture

| | |
|---|---|
| non-sustained release granules | 549.00 |
| sustained release granules | 200.00 |
| talc | 1.00 |
| Total Capsules | 750.00 |

In Example 23, the manufacture method was conducted analogously to the method outlined in Example 18.

EXAMPLE 24
mg

(a) Non-Sustained Release Granules

| | |
|---|---|
| epinastine hydrochloride | 10.00 |
| pseudoephedrine hydrochloride | 24.00 |
| methylephedrine hydrochloride | 24.00 |
| hydroxypropylcellulose | 12.59 |
| microcrystalline cellulose | 166.91 |
| lactose | 12.50 |
| Non-Sustained Release Granules Total | 250.00 |

(b) Sustained Release Granules

| | |
|---|---|
| pseudoephedrine hydrochloride | 36.00 |
| methylephedrine hydrochloride | 36.00 |
| hydroxypropylcellulose | 4.00 |
| sucrose | 79.00 |
| methacrylic acid copolymer, type B | 30.45 |
| magnesium stearate | 10.15 |
| glycerol esters of fatty acids | 3.10 |
| talc | 1.30 |
| Sustained Release Granules Total | 200.00 |

Compression Mixture

| | |
|---|---|
| non-sustained release granules | 250.00 |
| sustained release granules | 200.00 |
| microcrystalline cellulose | 126.00 |
| croscarmellose sodium | 12.00 |
| talc | 6.00 |
| magnesium stearate | 6.00 |
| Total | 600.00 |

In Example 24, the manufacture method was conducted analogously to the method outlined in Example 20.

EXAMPLE 25
mg

(a) Non-Sustained Release Granules

| | |
|---|---|
| epinastine hydrochloride | 10.00 |
| pseudoephedrine hydrochloride | 24.00 |
| methylephedrine hydrochloride | 24.00 |
| hydroxypropylcellulose | 12.59 |
| microcrystalline cellulose | 166.91 |
| lactose | 12.50 |
| Non-Sustained Release Granules Total | 250.00 |

(b) Sustained Release Granules

| | |
|---|---|
| pseudoephedrine hydrochloride | 36.00 |
| methylephedrine hydrochloride | 36.00 |
| hydroxypropylcellulose | 4.00 |
| sucrose | 79.00 |
| ammonio methacrylate copolymer | 30.45 |
| magnesium stearate | 10.15 |
| glycerol esters of fatty acids | 3.10 |
| talc | 1.30 |
| Sustained Release Granules Total | 200.00 |

Compression Mixture

| | |
|---|---|
| non-sustained release granules | 250.00 |
| sustained release granules | 200.00 |
| microcrystalline cellulose | 126.00 |
| croscarmellose sodium | 12.00 |
| talc | 6.00 |
| magnesium stearate | 6.00 |
| Total | 600.00 |

In Example 25, the manufacture method was conducted analogously to the method outlined in Example 20.

EXAMPLE 26
mg

(a) Non-Sustained Release Granules

| | |
|---|---|
| epinastine hydrochloride | 10.00 |
| pseudoephedrine hydrochloride | 24.00 |
| methylephedrine hydrochloride | 24.00 |
| hydroxypropylcellulose | 12.59 |
| microcrystalline cellulose | 166.91 |
| lactose | 12.50 |
| Non-Sustained Release Granules Total | 250.00 |

(b) Sustained Release Granules

| | |
|---|---|
| pseudoephedrine hydrochloride | 36.00 |
| methylephedrine hydrochloride | 36.00 |
| hydroxypropylcellulose | 4.00 |
| sucrose | 79.00 |
| ethylcellulose | 30.45 |
| magnesium stearate | 10.15 |
| glycerol esters of fatty acids | 3.10 |
| talc | 1.30 |
| Sustained Release Granules Total | 200.00 |

Compression Mixture

| | |
|---|---|
| non-sustained release granules | 250.00 |
| sustained release granules | 200.00 |
| microcrystalline cellulose | 126.00 |
| croscarmellose sodium | 12.00 |
| talc | 6.00 |
| magnesium stearate | 6.00 |
| Total | 600.00 |

In Example 26, the manufacture method was conducted analogously to the method outlined in Example 20.

1. A pharmaceutical composition comprising:
(a) an antihistaminically-effective amount of epinastine or a pharmaceutically acceptable salt thereof;
(b) a decongestant-effective amount of pseudoephedrine or a pharmaceutically acceptable salt thereof;
(c) methylephedrine or a pharmaceutically acceptable salt thereof, and
(d) a pharmaceutically acceptable carrier or excipient.
wherein the composition does not comprise Belladonna.

2. The pharmaceutical composition according to claim 1, wherein the pharmaceutical composition comprises 2 mg to 25 mg of epinastine or a pharmaceutically acceptable salt thereof.

3. The pharmaceutical composition according to claim 1, wherein the pharmaceutical composition comprises 10 mg to 240 mg of methylephedrine or a pharmaceutically acceptable salt thereof.

4. The pharmaceutical composition according to claim 2, wherein the pharmaceutical composition comprises 10 mg to 240 mg of methylephedrine or a pharmaceutically acceptable salt thereof.

5. The pharmaceutical composition according to claim 1, wherein the pharmaceutical composition comprises 10 mg to 300 mg of pseudoephedrine or a pharmaceutically acceptable salt thereof.

6. The pharmaceutical composition according to claim 2, wherein the pharmaceutical composition comprises 10 mg to 300 mg of pseudoephedrine or a pharmaceutically acceptable salt thereof.

7. The pharmaceutical composition according to claim 3, wherein the pharmaceutical composition comprises 10 mg to 300 mg of pseudoephedrine or a pharmaceutically acceptable salt thereof.

8. The pharmaceutical composition according to claim 1, wherein the active ingredients (a), (b), and (c) are formulated for instant release.

9. The pharmaceutical composition according to claim 1, wherein epinastine or a pharmaceutically acceptable salt thereof is formulated for instant release and at least a portion of the methylephedrine or a pharmaceutically acceptable salt thereof or the pseudoephedrine or a pharmaceutically acceptable salt thereof is formulated for sustained release.

10. The pharmaceutical composition according to claim 9, wherein the total amounts of the methylephedrine or a pharmaceutically acceptable salt thereof or the pseudoephedrine or a pharmaceutically acceptable salt thereof are formulated for sustained release.

11. The pharmaceutical composition according to claim 1, wherein the amounts of pseudoephedrine and methylephrine are the same.

12. The pharmaceutical composition according to claim 1, wherein the pharmaceutical composition is a bilayer tablet.

13. The pharmaceutical composition according to claim 9, wherein the pharmaceutical composition is a bilayer tablet.

14. The pharmaceutical composition according to claim 10, wherein the pharmaceutical composition is a bilayer tablet.

15. The bilayer tablet according to claim 12, comprising:
(a) a first layer A, providing for the sustained release of pseudoephedrine and methylephrine or the corresponding pharmaceutical salts thereof; and
(b) a second layer B, providing for the immediate release of epinastine, comprises an antihistaminically-effective amount of epinastine or a pharmaceutically acceptable salt thereof.

16. The bilayer tablet according to claim 12, further comprising a tablet coating C consisting of pharmaceutically acceptable excipients.

17. The bilayer tablet according to claim 13, further comprising a tablet coating C consisting of pharmaceutically acceptable excipients.

18. The bilayer tablet according to claim 14, further comprising a tablet coating C consisting of pharmaceutically acceptable excipients.

19. The bilayer tablet according to claim 15, further comprising a tablet coating C consisting of pharmaceutically acceptable excipients.

20. The bilayer tablet according to claim 15, wherein the first layer A comprises a decongestant-effective amount of pseudoephedrine or a pharmaceutically acceptable salt thereof, and methylephrine or a pharmaceutically acceptable salt thereof, in a matrix of a swellable hydrophilic polymer.

21. The pharmaceutical composition according to claim 1, wherein the pharmaceutical composition is a capsule.

22. The pharmaceutical composition according to claim 8, wherein the pharmaceutical composition is a capsule.

23. The pharmaceutical composition according to claim 9, wherein the pharmaceutical composition is a capsule.

24. The pharmaceutical composition according to claim 10, wherein the pharmaceutical composition is a capsule.

25. The capsule according to one of claims 21 to 24, wherein the capsule material comprises a compound selected from the group consisting of chitosan and starch, grain powder, oligosaccharides, methacrylic acid-methylacrylate, methacrylic acid-ethylacrylate, hydroxypropylmethylcellulose acetate, hydroxypropylmethylcellulose succinate, hydroxypropylmethylcellulose phthalate, polyvinyl alcohol, water-soluble non-toxic thermoplasts, hydroxypropylmethylcellulose, methylcellulose, hydroxypropylcellulose, hydroxypropyl starch, sodium alginate, gelatine, hard gelatine, and pullulan.

26. The capsule according to one of claims 21 to 24, wherein the ingredients are formulated as sustained release and non-sustained release granules.

27. The capsule according to claim 26, wherein the non-sustained granules are coated with a coating comprising a water insoluble polymer, intestinally soluble polymer, paraffin wax, higher alcohol, higher fatty acid, or higher fatty acid ester.

28. The pharmaceutical composition according to claim 1, wherein the ingredients are formulated as granules which are compressed to a tablet.

29. The pharmaceutical composition according to claim 8, wherein the ingredients are formulated as granules which are compressed to a tablet.

30. The pharmaceutical composition according to claim 9, wherein the ingredients are formulated as granules which are compressed to a tablet.

31. The pharmaceutical composition according to claim 10, wherein the ingredients are formulated as granules which are compressed to a tablet.

32. The pharmaceutical composition according to one of claims 28 to 31, wherein the ingredients are formulated as sustained release and non-sustained release granules which are compressed to a tablet.

33. The pharmaceutical composition according to claim 32, wherein the non-sustained release granules are coated with a coating comprising a water insoluble polymer, intestinally soluble polymer, paraffin wax, higher alcohol, higher fatty acid, or higher fatty acid ester.

34. A method of treating seasonal allergic rhinitis, seasonal allergic conjunctivitis, allergic rhinitis, allergic congestion of the Eustachian tubes, other allergic origin diseases treated using antihistamine and decongestant drugs, or cough, cold and flu symptoms in a patient in need of such treatment, the method comprising administering to a patient the pharmaceutical composition according to one of claims 1, 8, 9, or 10.
